(19) United States
(12) Patent Application Publication
(10) Pub. No.: US 2023/0001073 A1
(43) Pub. Date: Jan. 5, 2023

(54) ARRANGEMENT OF OPERATING ELEMENTS AND DISPLAYS IN A MEDICAL PUMP SYSTEM

(71) Applicant: B. Braun Melsungen AG, Melsungen (DE)

(72) Inventors: Jan Schwarz, Melsungen (DE); Maria Buerger, Malsfeld (DE); Christoph Erlen, Kassel (DE); James Richard Green, Chester (GB); Norbert Koop, Rellingen (DE); Jan Sokoll, Nortorf (DE); Stefan Espenhahn, Hamburg (DE); Joachim Schuetz, Fulda (DE)

(21) Appl. No.: 17/851,141

(22) Filed: Jun. 28, 2022

(30) Foreign Application Priority Data
Jul. 1, 2021 (DE) 20 2021 103 540.4

(51) Int. Cl. A61M 5/14; A61M 5/142; A61M 5/145

(52) U.S. Cl. CPC A61M 5/1413; A61M 5/14232; A61M 5/1452; A61M 2205/505; A61M 2205/18; A61M 2205/3306; A61M 2205/583

(57) ABSTRACT

A medical pump system includes at least one medical fluid pump of a first type and at least one medical fluid pump of a second type. The medical fluid pumps can be assembled or arranged vertically, with one of the medical fluid pumps positioned above the other. Each medical fluid pump has a movable front flap with an integrated display and operating elements. The medical fluid pumps can be directly connected to one another in a vertical arrangement, or arranged in a vertical arrangement with the aid of a carrying or holding device. When the medical fluid pumps are vertically arranged, the displays and operating elements of the respective medical fluid pumps are aligned vertically.

### CROSS-REFERENCE TO RELATED APPLICATION

This application claims priority under 35 U.S.C. § 119 to German Application No. 20 2021 103 540.4, filed Jul. 1, 2021, the content of which is incorporated by reference herein in its entirety.

### FIELD

The disclosure relates to an arrangement of the operating elements and displays in a medical pump system comprised of at least two fluid pumps of different types.

### BACKGROUND

Known fluid pumps have display units or displays or screens that serve as a graphical user interface. In addition to the display units, operating elements in the form of separately formed buttons are conventionally provided. Via the display unit as well as the operating elements, a user can enter instructions to the fluid pump and information about the fluid pump can be displayed to him. For example, the fluid flow rate and a dosing rate of the conveyed fluid can be set via the display unit itself or via function keys or operating elements coupled thereto. Information that can be represented via the display unit includes, inter alia, fluid pump parameters such as the conveying rate or dosing rate of the fluid, the fluid volume conveyed by the fluid pump, or the like.

Basically, medical devices comprising a display unit are known from the prior art. EP 2 973 100 B1 discloses, for example, an extracorporeal blood treatment system having a plurality of pumps and a display including a graphical user interface via which, among other things, conveying or dosing rates of a plurality of fluid reservoirs of the individual pumps are displayed and also function as control surfaces for a user via which he can perform certain settings on the extracorporeal blood treatment system.

Furthermore, a medical fluid pump comprising a display is known from CN 1 12 295 044 A. Via function keys formed separately next to the display, a user can set or adjust setting parameters of the fluid pump, which are reproduced via the display. In addition, the display can show further information about the operating history of the fluid pump.

Conventionally, medical fluid pumps are also used in combination as a pump system, in which multiple fluid pumps are mounted in a stacked manner or fastened within a carrying or holding device configured to hold the fluid pumps, whereby the medical fluid pumps are neatly bundled together during operation. However, in the event that medical fluid pumps of different types are used, the displays as well as the operating elements of the individual fluid pumps are not coordinated with each other, resulting in possible confusion or lack of clarity in the operation process for the operator.

### SUMMARY

Therefore, the present disclosure is based on the object of avoiding or at least mitigating the disadvantages described above. In particular, the present disclosure is intended to provide medical fluid pumps of various types having display units as well as operating elements which are coordinated with one another and enable the user/operator of the fluid pumps in a pump system to operate them more easily and clearly.

Accordingly, the disclosure relates to a medical pump system/pump interconnection comprising at least one medical fluid pump/infusion pump of a first type and at least one medical fluid pump/infusion pump of a second type, wherein the medical fluid pumps are arranged vertically one above the other and each have a movable front flap/housing flap with an integrated display/screen as well as operating elements/function keys. In this case, the displays as well as the operating elements with identical function are arranged in the assembled state of the medical pump system in alignment/straight/in a line one above the other via a direct connection of the fluid pumps to one another or with the aid of a carrying/arranging or holding device.

In other words, the at least two types of the medical fluid pumps have displays and operating elements that are designed and arranged in such a way that they are adapted to each other when combined to form a pump system and are arranged in the same places with respect to the individual levels of the fluid pumps. Thus, the displays as well as the operating elements with the same function of each of the fluid pumps are always located on a vertical imaginary line. It does not matter whether the medical fluid pumps are directly connected to each other or whether they are mounted within a special carrying or holding device.

This ensures that there will be no confusion between the operating elements of different fluid pumps, thus minimizing the risk of incorrect actuation of operating elements. In addition, the setting parameters presented via the displays can be quickly and easily distinguished from the other presented information for medical fluid pumps of different types.

In another preferred aspect, the medical fluid pump of the first type of the medical pump system according to the disclosure is a syringe pump and the medical fluid pump of the second type of the pump system according to the disclosure is a peristaltic pump.

In other words, the aligned orientation of the displays and the operating elements is ensured in a medical pump system of preferably at least one syringe pump as well as at least one peristaltic pump, although the modes of operation and thus the internal structure of these two types of medical fluid pumps differ greatly. Here, the syringe pump has a drive head as well as a syringe bracket, which are mounted on one side of the fluid pump display. Both components are used to automatically advance a syringe, which is held by these/on these components. In contrast, the peristaltic pump does not have any of these externally mounted components, which means that the housing of the peristaltic pump does not need to be adapted to them. Instead, the peristaltic pump preferably has an ejector button/eject button for the front flap of the fluid pump, whereby, for example, the front of the peristaltic pump is formed to be larger than the front of the syringe pump.

Preferably, the front flaps of the medical fluid pumps of the pump system according to the disclosure each have LEDs for status indication, which are also arranged in alignment one above the other in the assembled state of the medical pump system.

In other words, additional LEDs/lights are formed as a status indication/information display on the front flap of the medical fluid pumps. These are preferably arranged in parallel along the short side of the front flap and indicate, for example, whether the respective medical fluid pump is charged/operated via a power supply unit, or they issue warnings to the operator/user. These LEDs are aligned on the fluid pumps in such a way that they are aligned within a single vertical line when the fluid pumps are mounted as a medical pump system. Thus, it is not necessary for the user to reorient or search for the appropriate status indications when reading information from fluid pumps of different types that are arranged one above the other.

In another preferred aspect, the front flaps of the medical fluid pumps of the medical pump system according to the disclosure each have at least one light sensor.

In other words, additional light sensors are provided next to/on the displays of the medical fluid pumps, which detect the ambient brightness. As a result, the brightness of the displays and preferably the brightness of the LEDs for status indication of the medical fluid pumps can be adapted to the ambient brightness, which, on the one hand, ensures the recognizability of the displayed information and, on the other hand, ensures efficient energy utilization.

In another preferred aspect of the pump system according to the disclosure, the operating elements of the medical fluid pumps comprise at least a home button, an On/Off button and a stop button, which are arranged preferably to the left of the display of the fluid pumps.

In other words, the medical fluid pumps of different types have a group of operating elements/application buttons/function keys which are located on the same side of the display, preferably on the left. Thus, these operating elements for the medical fluid pumps are grouped in the same location, ensuring easier operation for the user. This arrangement also prevents, for example, inadvertent actuation of the ejector button of a peristaltic pump, since this ejector button is attached so as to be separate and apart from the operating elements described above, preferably on the right edge of the display.

In another preferred aspect, the operating elements of the medical fluid pumps are configured as a membrane keypad.

An embodiment of the operating elements of the medical fluid pumps in the form of a membrane keypad keeps the manufacturing effort and thus the manufacturing costs low. In addition, such a membrane keypad is insensitive to fluids that occur during operation of the medical fluid pumps. In contrast, when using conventional keys, fluid can easily enter the interior of the fluid pumps, resulting in unintentional damage to the internal components.

### BRIEF DESCRIPTION OF THE DRAWING FIGURES

The disclosed subject-matter will now be explained in more detail by means of advantageous embodiments and with reference to the associated Figures. In this connection, the Figures are merely of a schematic nature and serve exclusively to aid in understanding of the disclosure. It should be noted that the features of the individual embodiments may be interchanged with one another and may occur in any combination.

FIG. 1 is a front view of a medical fluid pump of the first type (syringe pump).

FIG. 2 is a front view of a medical fluid pump of the second type (peristaltic pump).

FIG. 3 is a front view of a medical pump system consisting of a syringe pump and a peristaltic pump mounted directly together.

FIG. 4 is a front view of a medical pump system consisting of two syringe pumps and two peristaltic pumps which are mounted in a carrying or holding device.

### DETAILED DESCRIPTION

In the following, the present disclosure and advantageous embodiments will be described with reference to the Figures.

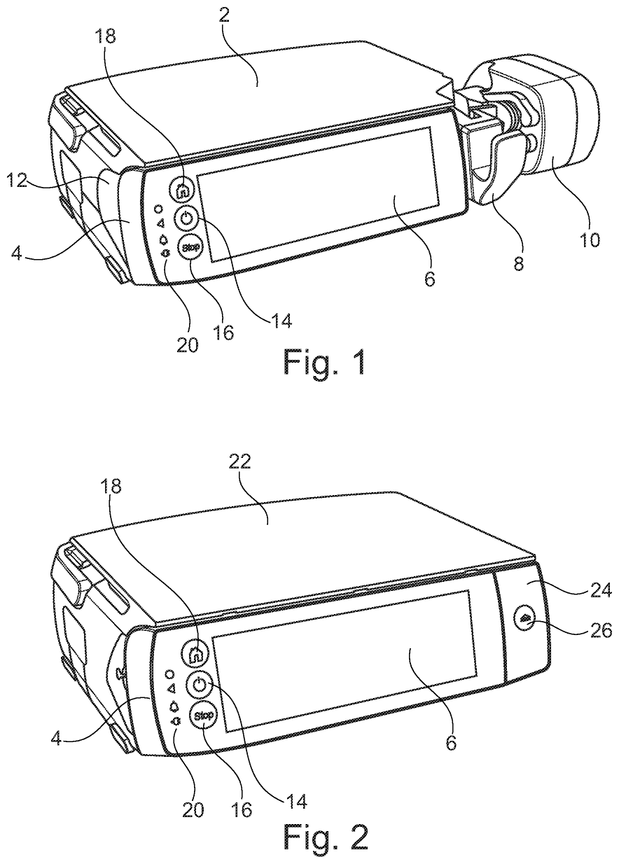

FIG. 1 is a front view of a syringe pump 2 as a medical fluid pump of the first type. The syringe pump 2 has a cuboid shape and has, as a front side, a front flap 4 with an integrated rectangular display 6. On one of its side surfaces (on the right in this front view), the syringe pump 2 has a syringe bracket 8 and a drive head 10, with the aid of which a syringe can be attached and compressed with a linear movement of the drive head 10 toward the display 6. The housing of the syringe pump 2 has a recess on one side due to the drive head 10 and the syringe bracket 8, i.e. the housing is designed such that the drive head 10 including the syringe bracket 8 can be attached to it. To insert the syringe, the front flap 4 is first folded around an axis on the lower side of the front flap 4 towards the bottom, and the syringe bracket 8, which is mounted between the display 6 and the drive head 10, is pulled out and then rotated through 90°, exposing the mounting location of the syringe. Clamping the syringe in/on the drive head 10, locking it via the syringe bracket 8 as well as closing the front flap 4 completes the process of mounting the syringe in the syringe pump 2. The front flap 4 can be opened via the recess 12. The front flap 4 has operating elements of the syringe pump 2 on one side of the display 6 (on the left in this front view), which include an On/Off button 14, a stop button 16 and a home button 18. These operating elements are arranged on a vertical line parallel to the left edge of the display. In addition, the front flap 4 has a row of LEDs for status indication 20 further to the left of the operating elements. These include, for example, a general alarm indication and an information indication about the status of the power supply connection. These LEDs for status indication 20 are also arranged one above the other and run parallel to the left (short) edge of the display 6.

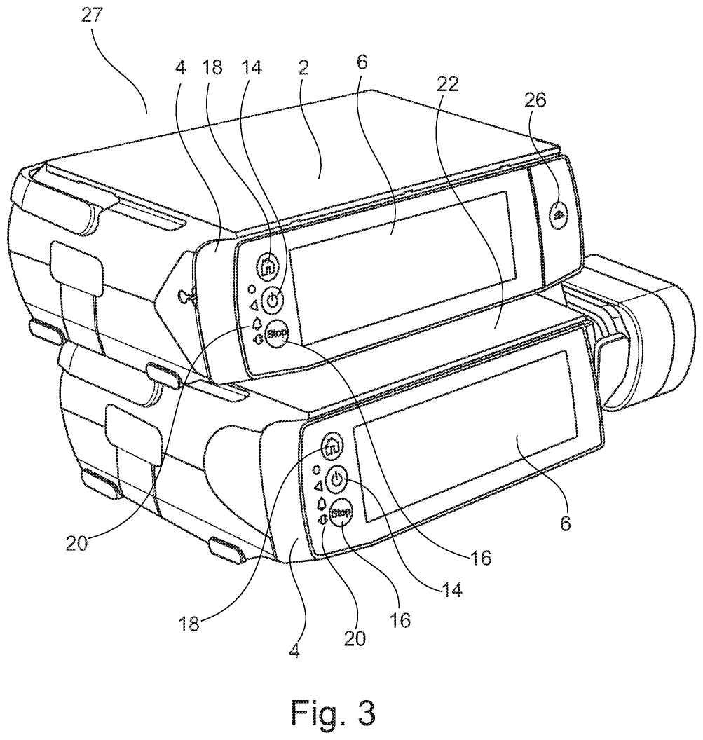

FIG. 2 is a front view of a peristaltic pump 22 as a medical fluid pump of the second type. The peristaltic pump 22 has the same cuboid shape as the syringe pump 2 of FIG. 1 and has, as a front side, a front flap 4 having an integrated rectangular display 6. The front flap 4 including the display 6 have the same dimensions as the front flap 4 and the display 6 of the syringe pump 2 from FIG. 1. Accordingly, the operating elements including the On/Off button 14, the stop button 16 and the home button 18 as well as the LEDs for status indication 20 are each arranged one above the other on an imaginary vertical line with, for example, a general alarm indication and an information indication about the status of the power supply connection. This line runs parallel along the short edge of the display 6. In contrast to the syringe pump 2 of FIG. 1, the peristaltic pump 22 in FIG. 2 has an additional housing portion 24 on the front side with an ejector button 26 on that side edge of the front flap 4 which is further away from the operating elements and the LEDs for status indication 20. Due to the fact that the peristaltic pump 22 does not have a drive head 10 and a syringe bracket 8, the housing of the peristaltic pump 22 is of continuous design and does not have a recess on either side. The front flap 4 is pivoted about an axis at the lower edge of the front flap 4 by actuation of the ejector button 26, whereby a replaceable tube can be inserted or removed for operation of the peristaltic pump 22.

FIG. 3 is a front view of a medical pump system 27 consisting of a syringe pump 2 and a peristaltic pump 22, which are directly connected to each other. The direct connection between the two fluid pumps of different types is achieved by means of a skid structure arranged on the housings of the fluid pumps. In this example, the skid structure formed on the bottom of the upper peristaltic pump 22 engages a guide structure on the top of the lower syringe pump 2. In this case, the skids as well as the corresponding guide structures are arranged on both of the sides that are perpendicular to the display 6. Due to the corresponding design of the individual fluid pumps as well as the skid or guide structures, the operating elements with an identical function as well as the LEDs for status indication 20 and the displays 6 are arranged in alignment one above the other in a directly connected state of the fluid pumps. This is especially true for a direct connection of fluid pumps of different types, for example for a medical pump system 27 consisting of syringe and peristaltic pumps 2, 22 with differently dimensioned front sides (see FIG. 3).

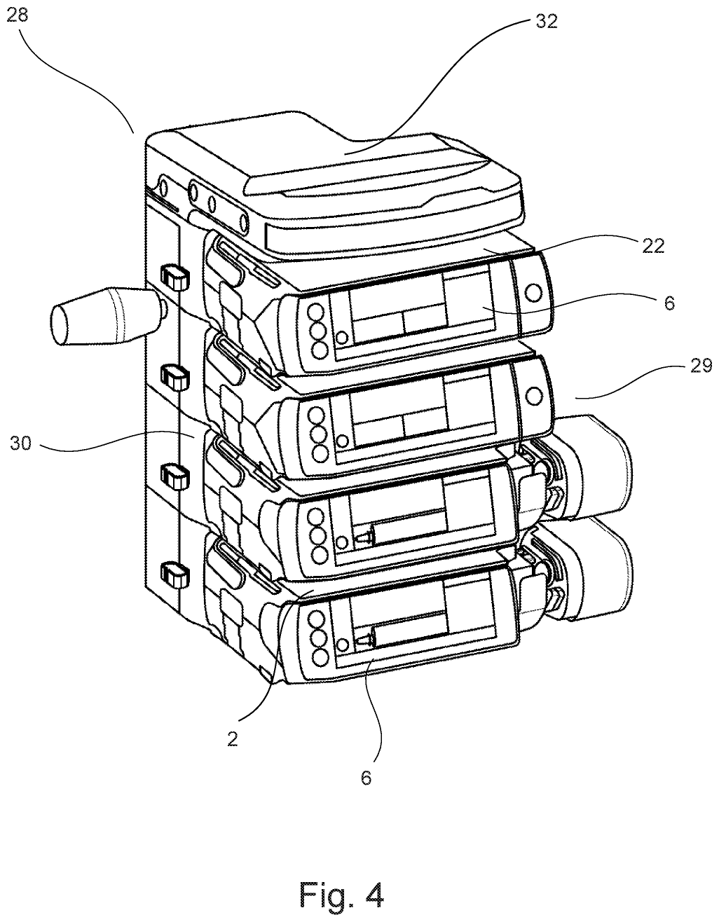

FIG. 4 is a front view of a medical pump system 29 consisting of two syringe pumps 2 and two peristaltic pumps 22 which are mounted in a carrying or holding device 28. The carrying or holding device 28 can be a shelf, a stacking system or a rack for medical fluid pumps and has an elongated base body 30 with a rectangular layout, on which the medical fluid pumps are mounted vertically one above the other. In addition, the base body 30 of the carrying or holding device 28 has four evenly vertically spaced trays on the front side which project vertically from said front side and are disposed between the fluid pumps mounted one above the other. The trays are formed as rectangular plates. The fluid pumps are fastened to/in the carrying or holding device 28 such that the back sides of the fluid pumps rest against the front of the base body 30 of the carrying or holding device 28 and are connected to the carrying or holding device 28 via a power supply unit and a communication interface. In addition, the carrying or holding device 28 has its upper side provided with a cover 32 which outputs central information about the mounted fluid pumps. The carrying or holding device 28 is designed such and adapted to the individual designs of the medical fluid pumps in such a way that the operating elements with an identical function and the displays 6 of the fluid pumps are arranged in alignment one above the other in a state mounted on the carrying or holding device 28. In particular, this applies to a medical pump system 29 consisting of syringe and peristaltic pumps 2, 22 with differently dimensioned front sides.

1. A medical pump system comprising at least two medical fluid pumps, the at least two medical fluid pumps comprising a first medical fluid pump of a first type and a second medical fluid pump of a second type, the first medical fluid pump being arranged vertically above the second medical fluid pump, each of the first medical fluid pump and the second medical fluid pump comprising a movable front flap with an integrated display and operating elements, the integrated display and operating elements of the first medical fluid pump being arranged in alignment with the integrated display and operating elements of the second medical fluid pump via: (1) a direct connection of the first medical fluid pump to the second medical fluid pump or (2) via a carrying or holding device.

2. The medical pump system according to claim 1, wherein the first medical fluid pump is a syringe pump and the second medical fluid pump is a peristaltic pump.

3. The medical pump system according to claim 1, wherein the movable front flaps each have LEDs as status indicators, and wherein the LEDs of the first medical fluid pump are arranged in alignment above the LEDs of the second medical fluid pump.

4. The medical pump system according to claim 3, wherein, on each of the first medical fluid pump and the second medical fluid pump, the LEDs indicate a general alarm and an information indication about a status of a power supply connection.

5. The medical pump system according to claim 1, wherein the movable front flaps each have at least one light sensor.

6. The medical pump system according to claim 1, wherein, on each of the first medical fluid pump and the second medical fluid pump, the operating elements comprise a plurality of buttons that include at least a home button, an On/Off button and a stop button, wherein the plurality of buttons are arranged on a left side of the display.

7. The medical pump system according to claim 6, wherein, on each of the first medical fluid pump and the second medical fluid pump, the plurality of buttons comprise an ejector button arranged on a right side of the display.

8. The medical pump system according to claim 6, wherein, on each of the first medical fluid pump and the second medical fluid pump, the home button, the On/Off button and the stop button are arranged on a vertical line.

9. The medical pump system according to claim 1, wherein the operating elements are provided on a membrane keypad.
